(19) United States
(12) Patent Application Publication
(10) Pub. No.: US 2017/0172723 A1
(43) Pub. Date: Jun. 22, 2017

(54) INTRALUMINAL DEVICE AND METHOD

(71) Applicant: BFKW, LLC, GRAND RAPIDS, MI (US)

(72) Inventors: James A. Foote, Ada, MI (US); Frederick J. Walburn, Grand Rapids, MI (US); Randal S. Baker, Grand Rapids, MI (US)

(73) Assignee: BFKW, LLC, GRAND RAPIDS, MI (US)

(21) Appl. No.: 15/406,126

(22) Filed: Jan. 13, 2017

Related U.S. Application Data
(62) Division of application No. 13/876,564, filed on Jun. 7, 2013.
(60) Provisional application No. 61/388,857, filed on Oct. 1, 2010.

(51) Int. Cl. A61F 2/04; A61F 5/00

(52) U.S. Cl. CPC A61F 2/04; A61F 5/0076; A61F 2002/044; A61F 2230/0067; A61F 2250/0039

(57) ABSTRACT

An intraluminal device and method includes positioning an intraluminal device in the recipient including positioning the intraluminal device in a lumen that experiences peristalsis. The intraluminal device has a surface defined by a wall. The surface is configured to generally conform to the shape and size of a portion of the lumen. The wall is adapted to reduce luminal spasm resulting from the peristalsis.

### BACKGROUND OF THE INVENTION

The present invention is directed to an intraluminal device and method and, in particular, to such a device and method that are useful in a lumen that is subject to peristalsis waves. The invention may be applied to a bariatric device and method of causing at least partial satiety in a recipient of the device and in particular to a bariatric device and method having an esophageal component. However, the invention may find various applications in other lumens that are subject to peristalsis.

Placement of an intraluminal device in a lumen that is subject to peristalsis may lead to stenosis or stricture of the underlying muscle.

### SUMMARY OF THE INVENTION

The present invention is directed to an intraluminal device and method that can reduce or eliminate stenosis or stricture from deployment of the device in a lumen that experiences peristalsis.

An intraluminal device and method, according to an aspect of the invention, includes positioning an intraluminal device in a lumen that experiences peristalsis. The intraluminal device has a surface defined by a wall. The wall is configured to generally conform to the shape and size of a portion of the lumen. The surface is adapted to reduce luminal spasm resulting from the peristalsis.

It is believed that the spasm results in micro-tears in the innermost tissue of the lumen which causes irritation and inflammation to the innermost tissue of a lumen. This, in turn, produces scaring or fibrosis, which can lead to stenosis or stricture. Accordingly, by reducing spasm resulting from the peristalsis, stenosis or stricture can be reduced.

The wall may be adapted to minimize progression of peristalsis in order to reduce or preclude a spasm. A stress may be applied with the surface on the lumen. The wall may have a transition zone at an end portion of the wall. A different stress may be applied on the lumen at the transition zone than inward of the transition zone. A greater stress may be applied at the transition zone than inward of the transition zone.

The wall may include an outwardly expanding internal mesh having a non-uniform cell structure. The cell structure may be less dense at the transition zone than inward of the transition zone, thereby defining the transition zone at least in part. A cover may be provided over the mesh. The cover may extend outward beyond the mesh thereby defining the transition zone at least in part.

The wall may define an edge portion that defines angulations therein. The angulations may be in the form of a bevel or a scalloped shape. The edge portion may be at a proximal end portion of the wall with respect to the peristalsis, at a distal end portion of the wall with respect to the peristalsis, or both.

The wall may be impregnated with an anti-spasm medication. The surface may be coated with an anti-spasm medication.

The surface may be configured to generally conform to the shape and size of a portion of (i) the esophagus, (ii) the intestine, (iii) the vagina, (iv) the bladder, (v) the urethra, (vi) the ureter, (vii) the fallopian tube, or (viii) the biliary duct. The intraluminal device may be used to treat (i) an anastomosis, (ii) a fistula, (iii) diverticular disease, (iv) a stomal opening, (v) an incision, and/or (vi) a stricture.

A bariatric device and method of causing at least partial satiety in a recipient, according to an aspect of the invention, includes positioning a bariatric device having an esophageal member in the recipient including positioning the esophageal member in the recipient's esophagus. The esophageal member has an esophageal surface defined by an esophageal wall. The esophageal surface is configured to generally conform to the shape and size of a portion of the esophagus. Receptors are stimulated with the esophageal surface in order to influence a neurohormonal mechanism in the recipient sufficient to cause at least partial satiety by augmenting fullness caused by food and simulating fullness in the absence of food. The esophageal wall is adapted to reduce esophageal spasm.

The esophageal wall may be adapted to minimize progression of peristalsis in order to reduce or preclude a spasm. The esophageal wall may have a transition zone at an end portion of the wall such that a different stress is applied on the esophagus at the transition zone than inward of the transition zone. This may include applying a lesser stress at the transition zone than inward of the transition zone. The esophageal wall may include an outwardly expanding internal mesh having a non-uniform cell structure. The mesh cell structure may be less dense at the transition zone than inward of the transition zone defining the transition zone at least in part. The esophageal member may include a cover over the mesh with the cover extending outward beyond the mesh thereby defining the transition zone at least in part.

The esophageal wall may define an edge portion, with the proximal edge portion defining angulations therein with the angulations minimizing the spasm of the lumen. The angulations may be in the form of a bevel or may be in the form of a scalloped shape. The edge portion may be at a proximal end portion of the wall with respect to the peristalsis, at a distal end portion of the wall with respect to the peristalsis, or both.

The esophageal wall may be impregnated with an anti-spasm medication or other medication. The esophageal surface may be coated with an anti-spasm medication.

The bariatric device may further include a cardiac member that is positioned at the cardiac region in the recipient's stomach. The cardiac member has a cardiac wall defining a cardiac surface that is configured to generally conform to the shape and size of a portion of the cardiac region of the stomach and stimulates receptors in order to influence a neurohormonal mechanism in the recipient sufficient to cause at least partial satiety by augmenting fullness caused by food and simulating fullness in the absence of food. The bariatric device may include a connector connected with the esophageal member and the cardiac member.

These and other objects, advantages and features of this invention will become apparent upon review of the following specification in conjunction with the drawings.

### BRIEF DESCRIPTION OF THE DRAWINGS

FIG. 1 is a side elevation of an intraluminal device and method according to an embodiment of the invention;

FIG. 2 is the same view as FIG. 1 with the cover removed to review an internal mesh;

FIG. 3 is a perspective view of an alternative embodiment of an intraluminal device and method according to an embodiment of the invention;

FIG. 4 is a side elevation of the intraluminal device in FIG. 3;

FIG. 5 is the same view as FIG. 4 of an alternative embodiment thereof;

FIG. 6 is a perspective view of another alternative embodiment of an intraluminal device and method according to an embodiment of the invention;

FIG. 7 is a side elevation of the intraluminal device in FIG. 6;

FIG. 8 is the same view as FIG. 8 of an alternative embodiment thereof;

FIG. 9 is a perspective view of a bariatric device and method according to an embodiment of the invention;

FIG. 10 is a side elevation of the bariatric device and method in FIG. 9; and

FIG. 11 is the same view as FIG. 1 of an alternative embodiment thereof.

### DESCRIPTION OF THE PREFERRED EMBODIMENT

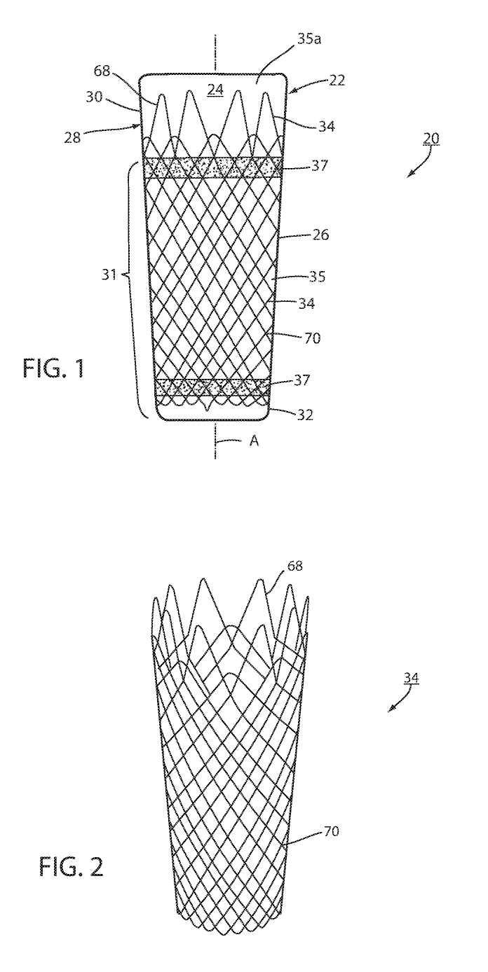

Referring now to the drawings and the illustrative embodiments depicted therein, an intraluminal device 20 includes an intraluminal member, such as an esophageal member, 22 having a surface 24 that is defined by a wall 26 having opposite end portions made up of a proximal portion 30 and a distal end portion 32 (FIGS. 1 and 2). Surface 24 is configured to generally conform to the shape and size of a portion of the lumen (not shown) of the recipient in which it is to be deployed. In particular, surface 24 is configured to generally conform to the shape and size of a portion of a lumen that experiences peristalsis. Examples of such a lumen include the esophagus, the colon, other portions of the intestines, ureter, urethra, biliary duct, fallopian tube, vas deferens, and the like. End portions 30, 32 are spaced apart along an axis A, which is the direction of peristaltic movement along the lumen in which device 20 is deployed. Wall 26 is defined by a support structure, such as a wire mesh 34, made from Nitinol wire, or the like, and a cover 35 over support structure 34. In the illustrated embodiment, cover 35 is a form of silicone or other flexible biologically inert substance that is applied, for example, to about 0.4 millimeter thickness. Cover 35 may have one or more overlapped layers at proximal end portion 30 and/or distal end portion 32. The layers of cover 35 are generally not adhered to each other where overlapped except at adhesive areas 37. This allows proximal end portion 30 and/or distal end portion 32 to be more pliant, which enhances a transition zone 28 between device 20 and the lumen in which it is deployed. Transition zone will be discussed in more detail below.

In one application, intraluminal device 20 is a bariatric device and member 22 is an esophageal member that is configured to generally conform to the shape and size of the distal portion of the esophagus and applies a stress to the esophagus. As disclosed in commonly assigned U.S. Pat. No. 7,846,174, the disclosure of which is hereby incorporated herein, such bariatric device stimulates receptors with surface 24 in order to influence a neurohormonal mechanism in the recipient sufficient to cause at least partial satiety by augmenting fullness caused by food and simulating fullness in the absence of food.

However, intraluminal device 20 may, alternatively, be configured to generally conform to the shape and size of a portion of other lumens, such as (i) the intestine, (ii) the vagina, (iii) the ureter, (iv) the urethra, (v) the biliary duct, (vi) the fallopian tube, and (vii) the vas deferens, by way of example, and may be used to treat conditions other than obesity, such as (i) an anastomosis, (ii) a fistula, (iii) diverticular disease, (iv) a stomal opening, (v) an incision, (vi) a stricture, and the like, as disclosed in U.S. Patent Application Publication No. 2008/0215076A1, the disclosure of which is hereby incorporated herein by reference.

Wall 26 is adapted to reduce or minimize spasm in the lumen of the recipient. This is accomplished by exerting a negative feedback mechanism to minimize spasm. While the precise mechanism by which the intraluminal device operates to reduce luminal spasm is not fully understood, it is believed that this may be accomplished by wall 26 being adapted to minimize progression of peristalsis in order to minimize or preclude the occurrence of a spasm. If peristalsis is not able to build up at surface 24, it will not become amplified to cause spasm which can create micro-tears in the mucosa or other underlying tissue. The micro-tears can become irritated or inflamed which, in turn, can lead to scarring or fibrosis formation. This can cause stenosis or stricture. Thus, by reducing spasm, stenosis and/or strictures can be reduced or eliminated. Thus, wall 26 may absorb peristalsis or reduce the intensity of luminal contractions. Alternatively, wall 26 may minimize circumferential contraction of the lumen. Thus, wall 26 may operate in a manner not related to peristalsis.

In one embodiment, wall 26 has a transition zone 28 that results in surface 24 applying different forces on the lumen at transition zone 28. In particular, surface 24 has a proximal end portion 30 and an inner portion 31 and a distal end portion 32. Transition zone 28 is at proximal end portion 30 in the illustrated embodiment. However, transition zone 28 could, alternatively, be at distal end portion, or both portions, 30, 32. Transition zone 28 applies a lower outward stress on the esophagus than inner portion 31 inward of proximal portion 30. This variability in force along surface 24 from transition zone 28 inwardly is believed to minimize progression of the peristalsis and, therefore, reduce spasm.

Wall 26 is formed in part by an outwardly expanding internal mesh 34. Mesh 34 may be formed by Nitinol, or the like, and may be formed by a weave of an elongated strand, by laser cut from a cylinder, or the like. Mesh 34 has a non-uniform cell structure. In particular, the cell structure has a less dense portion 68 at transition zone 28 than a more dense portion 70 at inner portion 31. Because mesh 34 produces at least in part the strain exerted by surface 24 on the lumen, the less dense cell structure at proximal portion 30 defines transition zone 28, at least in part. Cover 35 may have a portion 35a that extends proximally beyond mesh 34 thereby further defining transition zone 28, at least in part. Because there is no outwardly expanding mesh at portion 35a of cover 35, portion 35a will further exert less stress on the lumen than distal portion 31.

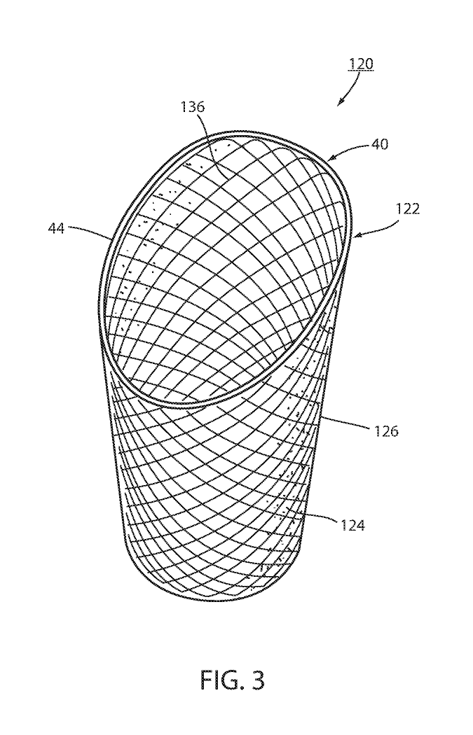

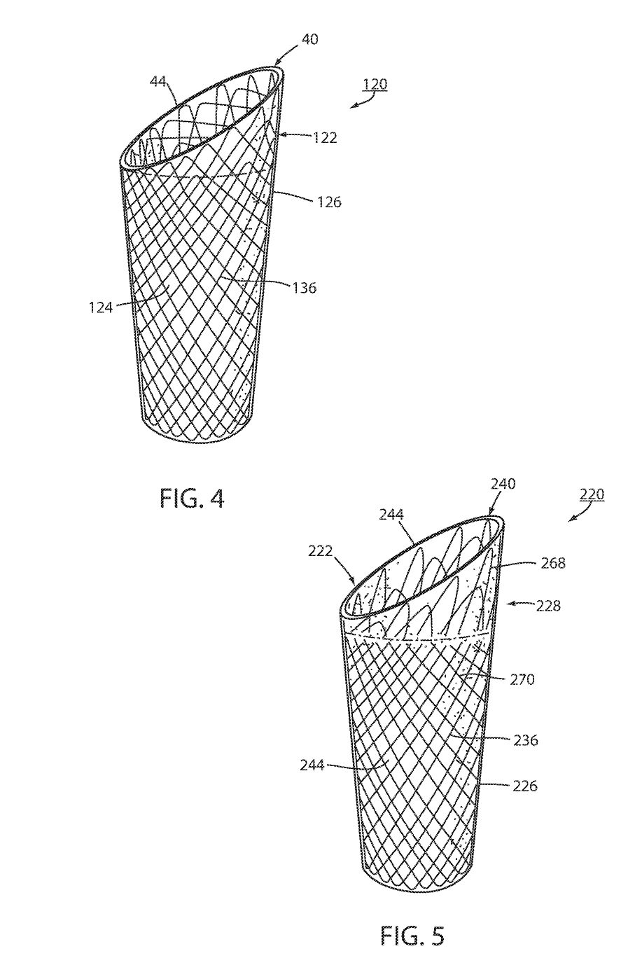

In an alternative embodiment, an intraluminal device 120 includes an intraluminal member 122 having a surface 124 defined by a wall 126 (FIGS. 3 and 4). Wall 126 may include an internal mesh 136 similar to mesh 36 and cover 129 over the mesh. Wall 126 defines an edge portion, such as proximal edge portion, 40 that provides an angulation in the wall. In particular, edge portion 40 defines an angulation in the form of a bevel 44. It is believed that such angulation minimizes luminal spasm in the recipient by breaking up the peristaltic wave so it cannot be amplified into a spasm.

It should be understood that various aspects illustrated herein can be combined. For example, an intraluminal device 220 includes an intraluminal member 222 having a surface 224 defined by a wall 226 (FIG. 5). Wall 226 defines a proximal edge portion 240 that defines an angulation in the wall, such as a bevel 244. In addition, wall 226 is made up of an internal mesh 236 that defines a transition zone 228 defined at least in part by mesh 236 having a cell structure that has a less dense portion 268 proximally than a more dense portion 270 distally. Therefore, intraluminal device 220 incorporates the principles of both intraluminal devices 20 and 120.

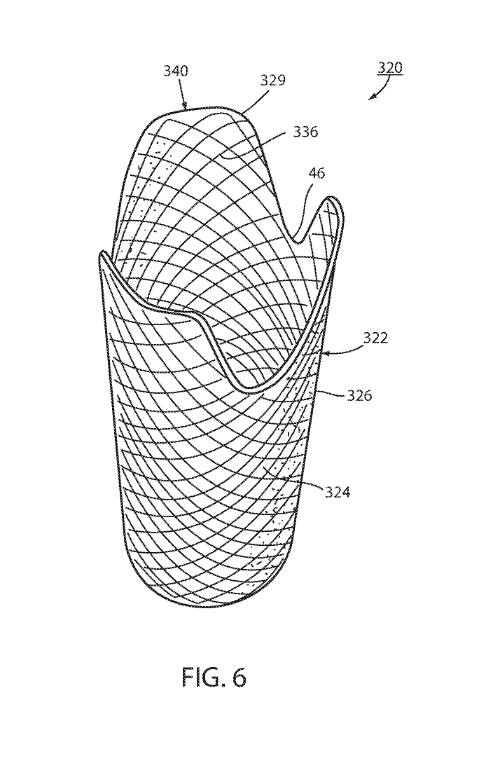

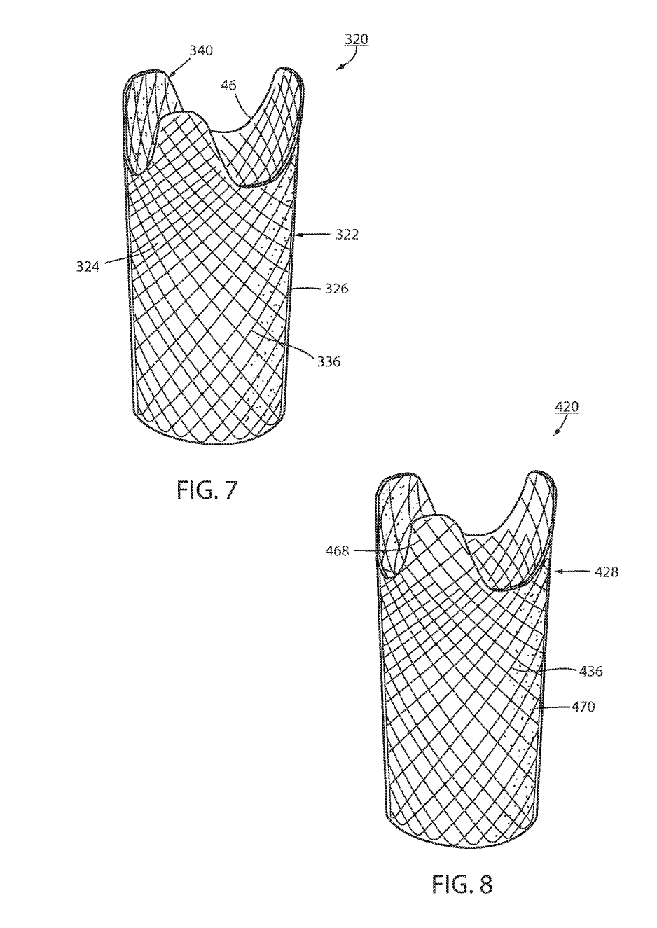

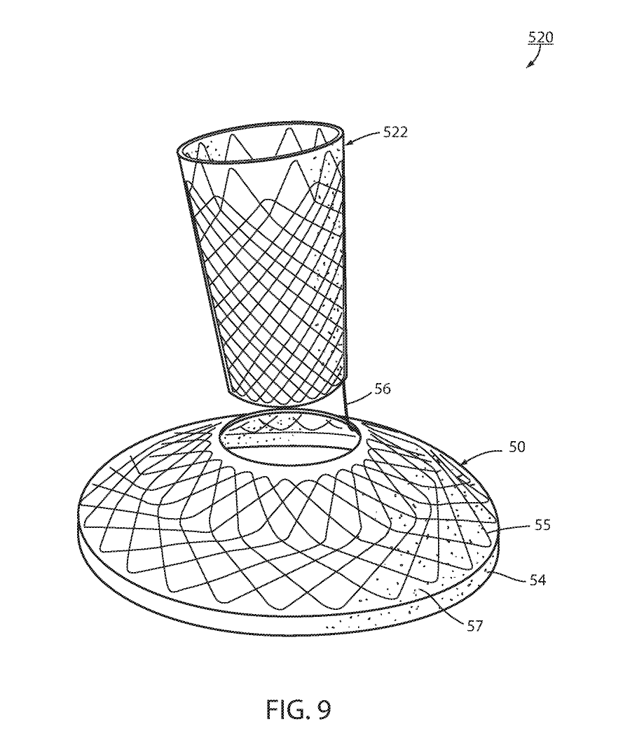

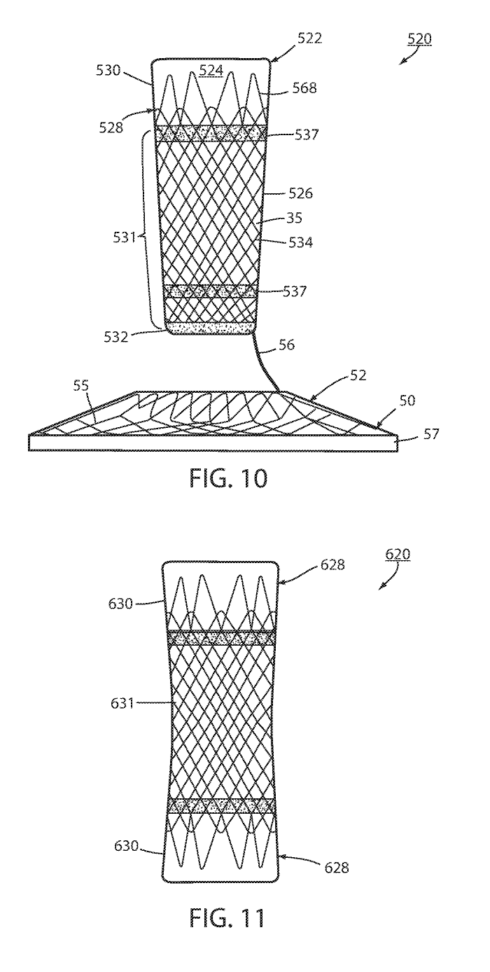

In another alternative embodiment, an intraluminal device 320 includes an intraluminal member 322 having a surface 324 defined by a wall 326 (FIGS. 6 and 7). Wall 326 may include an internal mesh 336 similar to mesh 36 and cover 329 over the mesh. Wall 326 defines a proximal edge portion 340 that provides an angulation in the wall. In particular, edge portion 340 defines an angulation in the form of a scalloped shape 46. In yet another alternative embodiment, an intraluminal device 420 is similar to intraluminal device 320, but includes an internal mesh 436 that defines a transition zone 428 made up at least in part by mesh 436 having a cell structure that has a less dense portion 468 proximally than a more dense portion 470 distally.

Other variations will be apparent to the skilled artisan. For example, esophageal wall 26, 126, 226, 326, 426 may be impregnated with an anti-spasm medication or other medication. Esophageal surface 24, 124, 224, 324, 424 may be coated with an anti-spasm medication or other medication.

In yet another alternative embodiment, an intraluminal device in the form of a bariatric device 520 includes an esophageal member 522, that may incorporate various features describe above. By way of example, esophageal member 522 has a surface 524 defined by a wall 526 having a proximal end portion 530 and a distal end portion 532. The surface is configured to generally conform to the shape and size of the distal portion of the esophagus. Wall 526 is defined by a support structure, such as a wire mesh 534, similar to mesh 34, and a cover 535, similar to cover 35. Cover 535 has overlapped portions at proximal and distal end portions 530, 532 that are joined together at respective adhesive areas 537. Mesh 534 has a less dense portion 568 that at least in part defines a transition zone 528. Cover 535 has a portion 535a that extends proximally beyond mesh 534, thereby further defining transition zone 528 at least in part. Transition zone 528 applies a lower outward stress on the esophagus than portion 531 of esophageal member 522 that is distal of transition zone 528.

Bariatric device 520 further includes a cardiac member 50 having a cardiac wall 54 defining a cardiac surface 52 that is configured to generally conform to the shape and size of a portion of the cardiac region of the stomach to stimulate receptors with cardiac surface 52 in order to influence a neurohormonal mechanism in the recipient sufficient to cause at least partial satiety by augmenting fullness caused by food and simulating fullness in the absence of food. Wall 54 is made up of a structural member, such as a mesh 55, that is formed of twisted overlapping loops of Nitinol wire, or the like, and a cover 57 made up of silicone or other flexible biologically inert substance that is applied to a thickness of approximately 0.4 millimeters. Bariatric device 520 may further include a connector 56 that is connected with esophageal member 522 and cardiac member 50 to thereby function in the manner set forth in U.S. Patent Application Publication No. 2010/0030017 A1, the disclosure of which is hereby incorporated herein by reference.

In yet another alternative embodiment illustrated in FIG. 11, an intraluminal device 620 includes a transition zone 628, similar to the transition zone 28 at both a proximal end portion 630 and a distal end portion 632 with respect to the peristalsis waves. Both transition zones 628 exert less stress on the lumen than a central portion 631.

While the foregoing description describes several embodiments of the present invention, it will be understood by those skilled in the art that variations and modifications to these embodiments may be made without departing from the spirit and scope of the invention, as defined in the claims below. The present invention encompasses all combinations of various embodiments or aspects of the invention described herein. It is understood that any and all embodiments of the present invention may be taken in conjunction with any other embodiment to describe additional embodiments of the present invention. Furthermore, any elements of an embodiment may be combined with any and all other elements of any of the embodiments to describe additional embodiments.

1-47. (canceled)

48. The method as claimed in claim 61 including applying a stress with said esophageal surface, wherein said reducing esophageal spasm includes said wall having a transition zone at an end portion of said wall, including applying a different stress on the esophagus at said transition zone than inward of said transition zone.

49. The method as claimed in claim 48 including applying a lower stress at said transition zone than inward of said transition zone.

50. The method as claimed in claim 49 wherein said wall includes an outwardly expanding internal mesh, said mesh having a non-uniform cell structure.

51. The method as claimed in claim 50 wherein said cell structure is less dense at said transition zone than inward of said transition zone thereby defining said transition zone at least in part.

52. The method as claimed in claim 51 wherein said esophageal member includes a cover over said mesh, said cover extending proximally beyond said mesh thereby defining said transition zone at least in part.

53. The method as claimed in claim 48 wherein said transition zone is at at least one chosen from a proximal end portion of said wall and a distal end portion of said wall.

54. The method as claimed in claim 61 wherein said esophageal wall having an edge portion, said edge portion defining angulations therein, said angulations minimizing spasm of the esophagus.

55. The method as claimed in claim 54 wherein said angulations comprise a bevel.

56. The method as claimed in claim 54 wherein said angulations comprise a scalloped shape.

57. The method as claimed in claim 54 wherein said edge portion is at a proximal end portion of said wall.

58. The method as claimed in claim 61 wherein at least one chosen from said esophageal wall and said cardiac wall is impregnated with an anti-spasm medication or at least one chosen from said esophageal surface and said cardiac surface is coated with an anti-spasm medication.

59. (canceled)

60. (canceled)

61. A method of causing at least partial satiety in a recipient, said method comprising:
positioning a device in the recipient, said device having an esophageal member, a cardiac member and a connector connected with said esophageal member and said cardiac member, including positioning the esophageal member in the recipient's distal esophagus and the cardiac member at the cardiac region in the recipient's stomach, said esophageal member having an esophageal surface defined by an esophageal wall that is configured to generally conform to the shape and size of a portion of the distal esophagus and said cardiac member having a cardiac wall defining a cardiac surface that is configured to generally conform to the shape and size of a portion of the cardiac region of the stomach; and
reducing stenosis or stricture of the esophagus of the recipient resulting from deployment of the device in the recipient.

62. A method of causing at least partial satiety in a recipient, said method comprising:
positioning a device in the recipient, said device having an esophageal member, a cardiac member and a connector connected with said esophageal member and said cardiac member, including positioning the esophageal member in the recipient's esophagus and the cardiac member at the cardiac region in the recipient's stomach, said esophageal member having an esophageal surface defined by an esophageal wall that is configured to generally conform to the shape and size of a portion of the esophagus and said cardiac member having a cardiac wall defining a cardiac surface that is configured to generally conform to the shape and size of a portion of the cardiac region of the stomach; and
reducing esophageal spasm of the recipient at said esophageal wall.

63. The method as claimed in claim 62 wherein said reducing build-up of peristalsis including applying a lower stress at a transition zone at a proximal end of said esophageal wall than inward of said transition zone.

64. The method as claimed in claim 63 wherein said esophageal wall includes an outwardly expanding internal mesh, said mesh having a non-uniform cell structure.

65. The method as claimed in claim 64 wherein said cell structure is less dense at said transition zone than inward of said transition zone thereby defining said transition zone at least in part.

66. The method as claimed in claim 65 wherein said esophageal member includes a cover over said mesh, said cover extending proximally beyond said mesh thereby defining said transition zone at least in part.

67. The method as claimed in claim 62 including another transition zone at a distal end portion of said wall and applying a different stress on the esophagus of the recipient at said another transition zone than inward of said another transition zone.

68. The method as claimed in claim 67 including applying a lower stress at said another transition zone than inward of said another transition zone.

69. The method as claimed in claim 62 wherein said esophageal wall having an edge portion, said edge portion defining angulations therein, said angulations minimizing spasm of the esophagus.

70. The method as claimed in claim 69 wherein said angulations comprise a bevel.

71. The method as claimed in claim 69 wherein said angulations comprise a scalloped shape.

72. The method as claimed in claim 69 wherein said edge portion is at a proximal end portion of said wall with respect to the peristalsis.

73. The method as claimed in claim 62 wherein at least one chosen from said esophageal wall and said cardiac wall is impregnated with an anti-spasm medication or at least one chosen from said esophageal surface and said cardiac surface is coated with an anti-spasm medication.

74. The method as claimed in claim 62 wherein said reducing esophageal spasm comprises reducing build-up of peristalsis of the esophagus of the recipient at said esophageal wall.

75. A method of causing at least partial satiety in a recipient, said method comprising:
positioning a device in the recipient, said device having an esophageal member, a cardiac member and a connector connected with said esophageal member and said cardiac member, including positioning the esophageal member in the recipient's distal esophagus and the cardiac member at the cardiac region in the recipient's stomach, said esophageal member having an esophageal surface defined by an esophageal wall that is configured to generally conform to the shape and size of a portion of the distal esophagus and said cardiac member having a cardiac wall defining a cardiac surface that is configured to generally conform to the shape and size of a portion of the cardiac region of the stomach; and
wherein said esophageal wall includes a transition zone at a proximal end portion, including applying a stress with said esophageal surface on the esophagus of the recipient including applying a different stress on the esophagus of the recipient at said transition zone than inward of said transition zone.

76. The method as claimed in claim 75 wherein said reducing build-up of peristalsis including applying a lower stress at said transition zone at a proximal end of said esophageal wall than inward of said transition zone.

77. The method as claimed in claim 76 wherein said esophageal wall includes an outwardly expanding internal mesh, said mesh having a non-uniform cell structure.

78. The method as claimed in claim 77 wherein said cell structure is less dense at said transition zone than inward of said transition zone thereby defining said transition zone at least in part.

79. The method as claimed in claim 78 wherein said esophageal member includes a cover over said mesh, said cover extending proximally beyond said mesh thereby defining said transition zone at least in part.

80. The method as claimed in claim 75 including another transition zone at a distal end portion of said wall and applying a different stress on the esophagus of the recipient at said another transition zone than inward of said another transition zone.

81. The method as claimed in claim 80 including applying a lower stress at said another transition zone than inward of said another transition zone.

82. The method as claimed in claim 75 wherein at least one chosen from said esophageal wall and said cardiac wall is impregnated with an anti-spasm medication or at least one chosen from said esophageal surface and said cardiac surface is coated with an anti-spasm medication.
